(19) United States
(12) Patent Application Publication
(10) Pub. No.: US 2003/0215935 A1
(43) Pub. Date: Nov. 20, 2003

(54) Apparatus and method for isolating living cells from an encapsulated organ tissue sample

(76) Inventor: David James Coon, Chapel Hill, NC (US)

Correspondence Address: OLIVE & OLIVE, P.A., 500 MEMORIAL STREET, PO BOX 2049, DURHAM, NC, 27702, US

(21) Appl. No.: 10/145,215

(22) Filed: May 14, 2002

(51) Int. Cl. C12M 3/00

(52) U.S. Cl. 435/284.1; 435/308.1; 435/268; 435/378

(57) ABSTRACT

An apparatus and method are disclosed for isolating living cells from an encapsulated organ or encapsulated organ tissue sample within a sealed environment. The apparatus and method utilize a sealed perfusion tank, a sealed perfusion box to create a sealed environment for the perfusion tank, and a filter cartridge containing one or more filters for separating the cells from the digested tissue sample.

### BACKGROUND OF THE INVENTION

1. Field of the Invention:

The field of the invention relates to the isolation of living cells. Specifically, the invention relates to an apparatus and method for isolating living cells from an encapsulated organ or encapsulated organ tissue sample within a sealed environment.

2. Description of Related Art:

The development of new medical procedures and new research avenues has led to a continuing need for improved methods and apparatus for isolating and collecting living cells from an encapsulated organ, such as a liver, kidney or spleen, of a human or animal donor. In certain types of cell therapy, for example, isolated living cells from a donor are injected into a patient or directly into a patient's affected organ. Those cells reside inside the patient's body and function normally to provide a therapeutic benefit to the patient. This technique is especially effective where the patient is experiencing decreased functioning or non-functioning of a particular organ. As an illustrative example, cell therapy is coming into increasing use in the treatment of diseases and injuries of the human liver. Other techniques are being developed for both kidney and spleen.

In liver cell therapy, healthy liver tissue is taken from an animal or human liver donor. In the case of removing liver cells from an animal, the process can be conducted on the living animal with the organ still in place, i.e. in situ. Human liver tissue, however, and some animal liver tissue, is removed from the body and processed outside the body. Because the liver tissue and in fact all living cells are very sensitive to pH, any solution used to process organ tissue to harvest living cells must be a buffer, that is, a solution with a pH adjusted to be compatible with living tissue. This pH is known to those skilled in the art, and ordinarily is approximately a pH of 7.2. The liver tissue is treated first with a chelating buffer, a buffer that removes cations, especially metal cations such as magnesium and calcium, and blood from the liver tissue, preferably a buffer containing EGTA. The liver is then treated with a digesting buffer that breaks apart the binding sites between the cells, preferably a buffer containing collagenase. The digested liver thereafter can be suspended in a collection buffer, a solution designed to sustain the life of, or nourish, the cells, preferably DMEM (Dulbeco's modified Eagle's medium). The digested liver tissue then is filtered to separate the desired cells from other organ material and the isolated cells are collected in the filtered collection buffer. This cellcontaining solution then is manipulated in ways known in the art, for example centrifugation, to separate the desired living cells from the solution.

Treatment is accomplished by taking, injecting, or implanting the collected living cells into a diseased or injured liver of a human patient or elsewhere as desired in such patient. The planted cells reside inside the patient where they act to assist and augment the functioning of the recipient's own diseased or injured liver. The functioning of the patient's liver is significantly enhanced by the presence of these donor cells, and the patient receives a distinct therapeutic benefit. Harvested progenitor or stem cells can be used for research purposes or for therapy that is not necessarily related to liver function.

The practicality of cell therapy treatment of the liver or any other encapsulated organ or body system obviously is dependent upon the availability of sufficient quantities of healthy cells from a donor. Currently, cells from encapsulated animal or human organs, such as the liver, are most often obtained by washing and digesting the organ tissue manually. The resulting digested tissue is then filtered through a series of filters of decreasing pore sizes until the cells are separated. Filtration can be done manually, one filter at a time, or can be done with a sealed system for accomplishing the separation through a series of filters.

Known technologies for collecting and isolating living cells involving manual handling are less than ideal. The digestion process is time consuming and labor intensive. The process also is best accomplished at roughly 37° C. to 42° C., which can be difficult to maintain. The cells can be damaged, killed or otherwise rendered incapable of performing their desired functions due to manual handling or incorrect temperatures. Typically, different methods and apparatus are used to harvest adult cells from those used to harvest progenitor cells; as a result, both types are not harvested from a single tissue specimen even though both types are present in that specimen. Moreover, known methods are performed almost entirely in open procedures. While stringent attempts usually are made to keep manual procedures under sterile conditions, at least where the cells are to be used for human therapy, the manual nature of the entire procedure easily leads to a compromise in cleanliness. Introduction of pathogens and contaminants into the system is a frequent problem.

A needs exists, therefore, for an apparatus and method for digesting cells from an encapsulated organ or encapsulated organ tissue sample and isolating the cells therefrom which is faster, less labor intensive and substantially reduces the potential damage to the cells. The needed apparatus and method should also be capable of simultaneously harvesting both adult and progenitor cells. Such needed apparatus and method should improve the ability to keep the process in a sterile environment, maintain a health environmental termperature, and to produce cells free of any other pathogen or contaminant.

### SUMMARY OF THE INVENTION

The present invention addresses the needs described above, as well as other problems, as can readily be seen from the description herein. The invention comprises a novel apparatus and method for digesting an encapsulated organ or an organ tissue sample from an encapsulated organ (hereinafter collectively referred to as the tissue sample) and for isolating living cells therefrom in a sealed and sterile system.

The apparatus of the invention includes a perfusion tank capable of being closed and holding the tissue sample and a buffer, and of allowing access to the tissue sample through a tank door, removable tank lid, or other tank access means. The perfusion tank also includes a waste drain, a perfusion tank cell drain, and a buffer recirculation connection.

The perfusion tank sits inside a perfusion box that contains the perfusion tank in a sealed environment during operation of the apparatus. The perfusion box allows the evacuation of buffers and the introduction of successive new buffers into the perfusion tank, and facilitates handling of the tissue samples in a sealed environment. The perfusion box has an access means such as a door. The tissue sample can be introduced through the door of the perfusion box and thence into the perfusion tank. When the perfusion box access means is closed, the perfusion tank and the tissue sample therein are contained in a sealed environment.

The apparatus further includes means for introducing, re-circulating, and discharging buffers into and from the perfusion tank through the buffer recirculation connection. A temperature control means senses and controls temperature so that the perfusion box, perfusion tank, tissue sample and associated buffers are maintained at a desired temperature. A pH control means senses the acidity of any buffer in the perfusion tank. This is accomplished by placing temperature and pH sensors in the buffer, connected to a reading device outside the perfusion box. The connection of sensors to readers can utilize known technologies for transmitting data wirelessly. Adjustments are made to the heater and to the pH of the buffering solution to maintain the desired temperature and pH levels, either manually or automatically using technologies known to those skilled in the art.

The perfusion tank cell drain passes from the bottom of the perfusion tank into the perfusion box via tubing or the like, and terminates at a filter cartridge having one or more filters for isolating the cells retrieved from the digested tissue sample while maintaining a sealed system. The filter cartridge can be located either inside the perfusion box or outside the perfusion box, so long as the drain system remains sealed. The filter cartridge has a filter cartridge discharge drain at the bottom for discharging fluid from the sealed filter enclosure and collecting cells after the larger particles have been removed. The filter cartridge drain can be outside the perfusion box when it is not critical that the cells be collected inside the sealed system for sterility purposes, or if sterility is otherwise achieved at the exterior filter cartridge drain location, for example with the use of sterile collection bags connected to the drain. The tubing from the tank and from the filter cartridge optionally can have one or more valves controlling flow from the perfusion tank.

In one embodiment of the invention, the filter cartridge includes a plurality of screening materials of decreasing pore size, usually in the range from about ten microns to three thousand microns in diameter. In one such embodiment, four filters having pore sizes ranging from about 1000 microns down to 125 microns in decreasing size are placed such that the digested organ passes from the largest mesh filter to the smallest until essentially only cells and fluid are removed from the filter cartridge drain.

In another embodiment of the invention, the perfusion box has work glove holes for use with sterile gloves or built-in work gloves for handling material used inside the perfusion box while maintaining sterility in the box.

The invention provides a novel apparatus and method for digesting tissue and isolating biological cells therefrom in a sealed environment. The invention makes this process quicker and less labor intensive with much less chance of contamination than previous methods used for this purpose. It also has been found that yields are improved and cells suffer less damage and retain greater function than cells isolated by previous methods and apparatus. Other features and advantages of the present invention will become apparent from the following detailed description of embodiments, taken with the drawings, which illustrate, by way of example, the principles of the invention.

### BRIEF DESCRIPTION OF THE DRAWINGS

FIG. 1 is a perspective view of the apparatus of the present invention.

FIG. 2 is an exploded view of the perfusion tank of the invention.

FIG. 3 is a cut-away view of the inside of a filter cartridge of the invention.

### DETAILED DESCRIPTION OF THE PREFERRED EMBODIMENTS

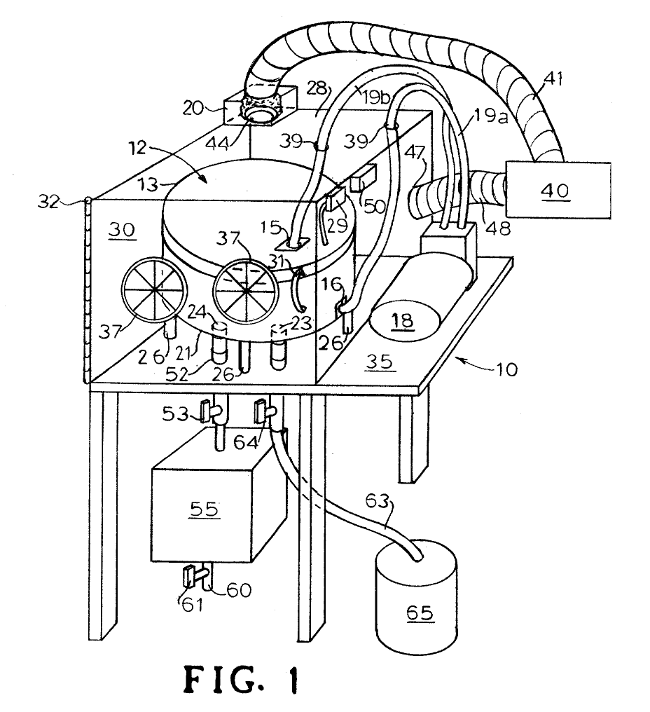

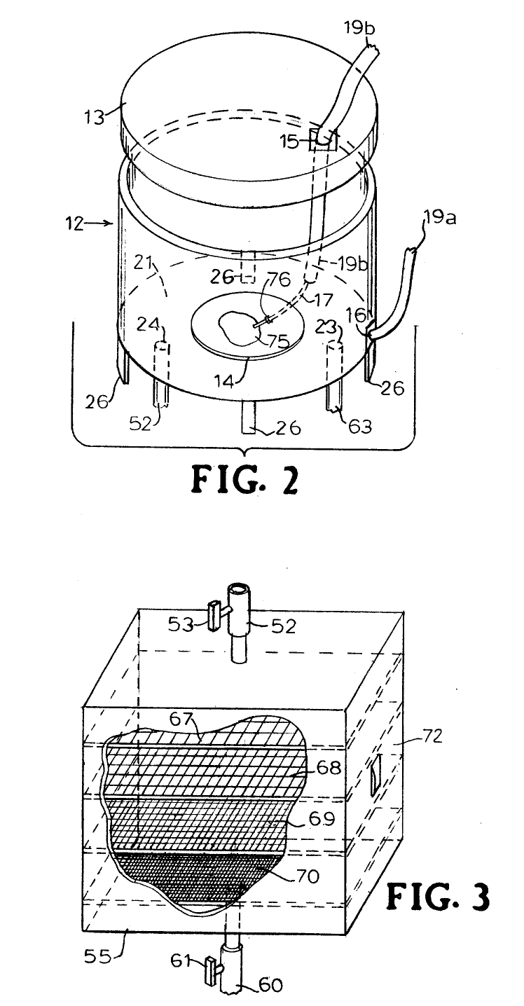

FIG. 1 depicts a perspective view of the apparatus 10 of the invention for use in the isolation of living cells from a tissue sample from an encapsulated organ such as a liver, kidney or spleen. As depicted, perfusion tank 12 is designed to hold the desired tissue sample. The tank can be made of any material used to contain or house sterile liquids and for medical sterile processes. For example, stainless steel, glass, or acrylic plastic could be used. In this embodiment, the perfusion tank 12 is cylindrical having a height (depth) of eight inches and a diameter of twelve inches. Lid 13 of perfusion tank 12 is removed and the desired tissue sample 75 (see FIG. 2) is placed within the perfusion tank 12 optionally on a tissue sample tray 14 (see FIG. 2).

A first end of a perfusion cannula 76 is inserted into a vein, artery, or other duct in the tissue sample 75. This can be done prior to placement in the perfusion tank 12, in the perfusion tank 12, or in the sealed environment of perfusion box 28. The duct of tissue sample 75 to which perfusion cannula 76 is connected must be of sufficient size to pass buffer into the tissue sample 75. Perfusion cannula connector 17 is connected to a second end of perfusion cannula 76.

A buffer (such as a chelating, digesting or collection buffer) is introduced into perfusion tank 12, typically by pouring sterilized buffer through the open top of perfusion tank 12. The amount of chelating or digesting buffer must be sufficient to adequately perfuse the tissue sample. For a liver tissue sample smaller than a complete adult human liver lobe, 500 ml of buffer typically is used. For an entire adult human liver, up to 4 liters of chelating or digesting buffer typically is used. The quantity of collection buffer must be sufficient to suspend the tissue sample contents (including the cells) in the collection buffer. Lid 13 is replaced after the buffer has been added.

Pump 18 is started and draws buffer from perfusion tank 12 through recirculation drain 16, which is connected to pump 18 via pump hose 19a. Preferably, pump 18 is a peristaltic pump which maintains the sealed environment of the buffer without contacting the pump 18 directly. The buffer is circulated via pump hose 19b into perfusion tank 12 through inlet 15 and continues through pump hose 19b to cannula connector 17, into cannula 76, thus circulating buffer through at least a portion of tissue sample 75. The buffer exits tissue sample 75 into perfusion tank 12 where the buffer may be recirculated for a desired time by pump 18 through tissue sample 75. A timer can be provided on pump 18 or the process can be computer controlled.

A flow meter also can be included in the pump system with flow rates of 15-150 mls/min being preferred. Perfusion tank 12 also can be fitted with a means for detecting the pH of the buffer, typically via a probe inserted into the buffer. The acidity of the buffer then can be observed and if desired can be adjusted, either manually or by other means, so that the pH of the buffer is maintained at a desired level.

Perfusion tank 12 is fitted in bottom area 21 with a waste drain 23 and a perfusion tank cell drain 24. In this embodiment, bottom area 21 preferably also has perfusion tank legs 26, for supporting perfusion tank 12 and providing an operator access to the area below bottom area 21. Alternately, no legs are provided and the operator gains access below bottom area 21 by moving perfusion tank 12 as needed.

Perfusion tank 12 is enclosed within perfusion box 28 which creates a sealed and preferably sterile environment. In this embodiment, being used by way of example, perfusion box 28 is constructed of a clear material, for instance, medical grade acrylic, glass, or polypropylene. Perfusion box 28 is fitted with a door 30 with handle 31 and door hinge 32. Door 30 may be the entire front side of perfusion box 28 as illustrated, but alternatively could be formed in an opening in any side of the perfusion box or in its floor or ceiling, or in a portion of any of those sides, floor or ceiling. Perfusion box 28 sits on table 35, which acts as a support means for the perfusion box 28 and perfusion tank 12. The perfusion box is sealed to table 35 so that a portion of table 35 serves as the floor of perfusion box 28. Alternatively, a separate perfusion box floor can be provided. When door 30 of perfusion box 28 is closed, perfusion box 28 forms a complete sealed compartment enclosing perfusion tank 12.

Perfusion box door 30 is fitted with glove holes 37 designed so that access may be had to the sealed perfusion box 28 without opening door 30 during the use of the entire apparatus 10. Preferably the glove holes contain preformed gloves or flexible plastic or the like that are sealed so that when hands are thrust through the glove holes they are contained within the preformed gloves or flexible plastic or the like and do not open a passageway for contamination into the sealed environment of perfusion box 28.

Perfusion tank 12 is heated to keep the contents, especially the tissue sample, at a particular desired temperature, preferably from about 37° C. to 42° C. This is accomplished in this embodiment by providing a heat source 40 which circulates heated air through heat tube 41, through air filter 20, through heat tube hole 44, and into perfusion box 28. A heat exit tube 48 is connected to heat exit 47 and directs the air back to heater 40. Temperature is monitored by placement of a temperature sensor 50 inside perfusion box 28. Using conventional temperature control mechanisms, the temperature inside is adjusted as necessary to obtain and maintain the desired temperature levels.

The air in perfusion box 28 preferably is recirculated and is filtered by a medically accepted process filter of suitable size and shape. In the preferred embodiment, a Class 100 HEPA filter is used. Filter 20 is shown sitting on top of perfusion box 28, although other placements would be equally efficacious to filter the air. If a forced air heating system is employed, as shown in FIG. 1, then the filter should filter the heated air at a point after it has exited heating unit 40.

Hose holes 39 in perfusion box 28 allow pump hoses 19a and 19b to exit and enter, respectively, the perfusion box 28. Pump hoses 19a and 19b exit and enter perfusion tank 12 through perfusion inlet 15 and recirculation drain 16 respectively. Perfusion tank cell drain 24 is connected to cell drain tube 52 which passes from perfusion tank bottom area 21 downward through cell tube valve 53 and table 35, and out of the sealed part of apparatus 10. The contents of cell drain tube 52 remain part of the sealed system, however. Cell tube valve 53, which controls flow from perfusion tank 12 to filter cartridge 55, preferably is located outside the sealed perfusion box 28 when filter cartridge 55 is located outside perfusion box 28. As later explained, digested material from the perfusion tank 12 runs through a series of filters in filter cartridge 55 (see FIGS. 1 and 3) and the cells which remain at the end of the filtration process are collected from filter cartridge drain 60. Flow from filter cartridge drain 60 is controlled by filter cartridge drain valve 61.

Waste drain 23 is connected to waste drain tube 63 (FIG. 1) which passes from the environment of the perfusion tank 12 through the sealed perfusion box 28, through table 35 to the outside environment. Waste tube valve 64, preferably located outside sealed perfusion box 28, controls the flow through waste drain tube 63. Waste drain tube 63 is terminated in waste container 65 outside the sealed environment of the apparatus 10.

FIG. 2, previously referred to, is an exploded view of perfusion tank 12. Perfusion tank lid 13 is shown removed from the perfusion tank 12. In the exploded view, tissue sample tray 14, preferably used to hold tissue sample 75, can be seen installed inside perfusion tank 12. Tissue sample 75 is shown with cannula 76 inserted therein and connected to cannula attachment 17. Other features of the perfusion tank can be seen including perfusion tank cell drain 24 and waste drain 23. Perfusion inlet 15 and recirculation drain 16 are more clearly seen in this view. Also shown are cell drain tube 52 and waste drain tube 63.

FIG. 3 is a cut-away view of filter cartridge 55. In this view, filters 67, 68, 69, and 70 are shown, and the successively smaller dimension of the pore sizes of these filters is illustrated. In this embodiment, the filter sizes are 1000 microns (filter 67), 500 microns (filter 68), 250 microns (filter 69), and 125 microns (filter 70). In this view, filter door 72 is on the right side of filter cartridge 55, enabling the removal of filters 67, 68, 69, and 70 upon completion of the process. Also shown are cell drain tube 52 and cell drain tube valve 53. Also shown are filter cartridge drain 60 and valve 61.

In practice, the apparatus and method of the invention are used to separate and remove isolated cells from a tissue sample. The process of using the apparatus and method of the invention begins by selecting a tissue sample from an encapsulated organ such as liver, kidney or spleen. Either outside of apparatus 10 or within the environment of perfusion box 28, tissue sample 75 located on sample tray 14 (FIG. 2) is prepared by placing perfusion cannula 76 into a vein, artery, or other duct of sufficient size to perfuse tissue sample 75 with buffer. Tissue sample 75 is inspected. All veins, arteries, and ducts, other than that into which perfusion cannula 76 is placed, are sealed using tape, glue, or other medically or biologically acceptable adhesive material. Any other portions of the exterior of the encapsulated organ tissue sample 75 that are not already sealed by the organ's capsule also are sealed. The prepared tissue sample 75 then is placed onto tissue sample tray 14 in perfusion tank 12. A second end of perfusion cannula 76 is connected to a first end of cannula connector 17. A second end of cannula connector 17 is connected to hose 19b. All desired sterile buffers are placed separately inside perfusion box 28 in individual containers for use in the process. In an alternate embodiment (not shown) buffers are introduced through lines connected to the perfusion tank 12 from storage tanks outside the sealed system. Door 30 to perfusion box 28 is closed. By reaching through glove holes 27, chelating buffer, preferably buffer containing EGTA, is introduced into perfusion tank 12 through the open top. Lid 13 is then closed. Assuming that the tissue sample, perfusion tank components, and interior of the perfusion box are sterile to begin with, use of sterile technique ensures that the system remains sterile during subsequent processing.

Pump 18 is turned on and chelating buffer from perfusion tank 12 is introduced into tissue sample 75 through cannula 76. Chelating buffer is allowed to recirculate through tissue sample 75, typically from 5 to 20 minutes, removing magnesium, calcium, and blood. When the desired time for circulating chelating buffer has elapsed, waste drain flow valve 64 is opened and the chelating buffer drained from the system out to waste container 65. Valve 64 is then closed. Next, a digesting buffer, preferably a buffer containing collagenase, is introduced into perfusion tank 12, typically in a quantity substantially equal to the quantity of chelating buffer that previously had been introduced. If the tissue sample is human liver, pump 18 is adjusted to circulate the digesting buffer through tissue sample 75 at a rate of 15-150 mls/min, for approximately 10 to 25 minutes. The rate is adjusted as necessary, preferably to keep the tissue visibly inflated during perfusion. The inflating of the tissue helps insure that all areas of tissue sample 75 are contacted by buffer. After tissue sample 75 has been sufficiently processed with the digesting buffer, the waste drain flow valve 64 is again opened and the digesting buffer is removed through waste drain 23 into waste container 65 and valve 64 is closed. Lastly, the tissue sample is opened (e.g., sliced open with a scalpel already placed in the sealed environment of perfusion box 28). A collection buffer, preferably DMEM, is introduced into perfusion tank 12 in a quantity sufficient to suspend the tissue sample contents (including the cells) in the collection buffer. The collection buffer, carrying the tissue sample contents, then is drained through perfusion tank cell drain 24 and through filters 67, 68, 69 and 70 in filter cartridge 55. The resulting suspension of filtered cells in collection buffer is removed through filter cartridge drain 60. The cells preferably are drained into sterile containers or a sterile transfer container if sterility is a concern. Individual cells may then be separated via centrifugation or other techniques known to those skilled in the art, and adult cells likewise may be separated from progenitor cells, if desired, using techniques known to those skilled in the art.

It is clear from the above that substitution in materials and methods could be known by one skilled in the art with the teachings of the invention and is therefore included in the contemplated invention.

What is claimed is:

1. An apparatus for recirculating buffer through and isolating living cells from an encapsulated organ or an encapsulated organ tissue sample comprising:
(a) a perfusion tank capable of holding said tissue sample and said buffer and of allowing access to said tissue sample during use, comprising a waste drain, a cell drain, and a buffer recirculation connection;
(b) a perfusion box for containing said perfusion tank in a sealed environment, comprising a container enclosing said perfusion tank and means for accessing said perfusion tank;
(c) means for recirculating said buffer in said perfusion tank and said tissue sample through said buffer recirculation connection; and
(d) a filter cartridge connected to said cell drain having at least one filter capable of separating said living cells from an effluent passing out of said cell drain.

2. An apparatus according to claim 1, wherein said perfusion box further comprises access to said perfusion via a pair of glove holes.

3. An apparatus according to claim 1, wherein said perfusion box further comprises access to said perfusion box via built-in gloves.

4. An apparatus according to claim 1, wherein said filter cartridge comprises a plurality of filters of decreasing pore size.

5. An apparatus according to claim 4 wherein said filter cartridge comprises four filters of decreasing pore size.

6. An apparatus according to claim 1, which further comprises a support means for said perfusion tank and perfusion box.

7. An apparatus according to claim 6 wherein said support means elevates said perfusion tank within said perfusion box.

8. An apparatus according to claim 1, which further comprises a temperature sensor for detecting the temperature within said perfusion box.

9. An apparatus according to claim 8, which further comprises temperature control means associated with said sensor for adjusting the temperature within said apparatus.

10. An apparatus according to claim 1, which further comprises a means for filtering the air within said perfusion box.

11. An apparatus according to claim 10, wherein said means for filtering said air is a HEPA air filter.

12. An apparatus according to claim 1, which further comprises means for measuring the pH of said buffer.

13. An apparatus according to claim 1, which further comprises a timer for controlling the time of at least one operation of the apparatus.

14. An apparatus according to claim 1, which further comprises a flow meter for monitoring the flow rate of said buffer through said tissue sample.

15. A method for isolating living cells from an encapsulated organ tissue sample, comprising:
(a) preparing said tissue sample for use with a buffer recirculating system;
(b) attaching said buffer recirculating system to said tissue sample;
(c) digesting said tissue sample by recirculating at least one buffer through said tissue sample in a closed environment with said buffer recirculating system, said buffer being selected to separate living cells from said tissue sample;
(d) maintaining the temperature of said closed environment at a desired temperature; and
(e) filtering said digested tissue sample in said closed environment to isolate the desired cells.

16. A method according to claim 15 wherein said closed environment comprises a perfusion tank for holding the tissue sample and said at least one buffer and a perfusion box for enclosing said perfusion tank.

17. A method according to claim 15 wherein said at least one buffer is recirculated by placing the buffer in the perfusion tank and recirculating said at least one buffer between the perfusion tank and the tissue sample with said buffer recirculating system.

18. A method according to claim 15 wherein said filtering is performed by a filter cartridge containing at least one filter.

19. A method according to claim 15 wherein the air inside said closed environment is filtered.

20. A method according to claim 15 wherein the air inside said closed environment is heated.

21. A method for isolating living cells from an encapsulated organ tissue sample comprising:
(a) establishing a perfusion tank capable of holding said tissue sample and a processing buffer and allowing access to said tissue sample during use, comprising a waste drain, a cell drain, and a buffer recirculation connection;
(b) establishing a perfusion box for containing said perfusion tank in a sealed environment, comprising a container enclosing said perfusion tank and a means for accessing said perfusion tank;
(c) circulating said processing buffer in said perfusion tank and within said tissue sample through said buffer recirculation connection;
(d) establishing a cartridge connected to said cell drain having one or more filters capable of separating said living cells from an effluent passing out of said cell drain; and
(e) separating said living cells by utilizing said cartridge.

22. An apparatus adapted for being used with an associated buffer source for isolating living cells from an encapsulated organ or organ tissue sample, comprising:
(a) a perfusion tank having means operative during use:
(i) for holding said tissue sample and a buffer;
(ii) for allowing access to said tissue sample;
(iii) for separately drawing waste and cells from said tank; and
(iv) connectable for buffer recirculating:
(b) a perfusion box enclosing said perfusion tank and having means providing access thereto;
(c) means connectable with said perfusion tank buffer recirculation means for recirculating buffer in said perfusion tank and tissue sample; and
(d) filter means connected to said perfusion tank means for separately draining cells and adapted for separating living cells from effluent received by said filter means.
